(19) United States
(12) Patent Application Publication
(10) Pub. No.: US 2010/0196923 A1
(43) Pub. Date: Aug. 5, 2010

(54) PLURIPOTENT ADULT STEM CELLS

(76) Inventor: Anthony Atala, Winston-Salem, NC (US)

Correspondence Address: MYERS BIGEL SIBLEY & SAJOVEC, PO BOX 37428, RALEIGH, NC, 27627, US

(21) Appl. No.: 12/673,306

(22) PCT Filed: Aug. 14, 2008
(86) PCT No.: PCT/US08/09721

§ 371 Date: Mar. 17, 2010

Related U.S. Application Data
(60) Provisional application No. 60/955,677, filed on Aug. 14, 2007.

(51) Int. Cl. G01N 33/53; C12N 5/074

(52) U.S. Cl. 435/7.1; 435/366; 435/377

(57) ABSTRACT

Disclosed herein are pluripotent adult stem cells and methods of use thereof. The cells are found in, or collected from, an adult tissue or fluid. In some embodiments, the cells are c-kit positive and SSEA-4 positive, and can he differentiated into multiple tissue types, e.g., adipogenic, osteogenic, myogenic, endothelial, neurogenic and hepatic tissues.

### RELATED APPLICATIONS

This application claims the benefit under 35 U.S.C. §119(e) of U.S. Provisional Patent Application Ser. No. 60/955,677, filed Aug. 14, 2007, the disclosure of which is incorporated herein by reference in its entirety.

### FIELD OF THE INVENTION

The present invention concerns the isolation, propagation and use of pluripotent adult stem cells.

### BACKGROUND OF THE INVENTION

Stem cells are unspecialized cells that self-renew for long periods through cell division, and can be induced to differentiate into cells with specialized functions.

These qualities give stem cells great promise for use in therapeutic applications to replace damages cells and tissue in conditions such as spinal cord injuries, diabetes, Alzheimer's disease and stroke.

Embryonic stem (ES) cells are derived from the blastocyst of an embryo and have the potential to develop into any type of cell (i.e., they are “totipotent”). ES cells tend to spontaneously differentiate into various types of tissues, and the control of their direction of differentiation can be challenging. They are also prone to the formations of tumors, or teratomas. Finally, there are unresolved ethical concerns that are associated with the destruction of embryos in order to harvest human ES cells. These problems limit their availability for research and therapeutic applications.

Adult stem (AS) cells are found among differentiated tissues. Stem cells obtained from adult tissues typically have the potential to form a more limited spectrum of cells (i.e., “multipotent”), and typically only differentiate into the cell types of the tissues in which they are found, though recent reports have shown some plasticity in certain types of AS cells. They also generally have a limited proliferation potential. The most extensively studied adult stem cells are the hematopoietic stem cells found in bone marrow and the neural stem cells found in brain tissues.

Amniotic fluid stem (AFS) cells of fetal origin have also been recently described (De Coppi et al. (2007) Nature Biotechnology 25(1):100-6; PCT Application WO 03/042405 to Atala and DeCoppi). These cells possess some of the characteristics of ES cells and can be guided to differentiate into many different cell types.

Stem cells are an attractive source of cells for therapeutic applications, particularly in treatments involving cell replacement therapies. For example, neurodegenerative disorders such as Parkinson's disease may be treated with stem cells that are differentiated into dopaminergic neurons for cell replacement therapy. Stem cells obtained from adult tissues, or adult stem cells, are particularly desirable because they offer the ability to more readily use cells taken from the very subject to be treated, minimizing an immune rejection of transplanted or grafted cells: However, most stem cells that have been isolated from adult tissues thus far show only a limited ability to proliferate and to form the various types of differentiated cells of the body, which limits their therapeutic applications. Therefore alternative sources of adult stem cells are needed.

### SUMMARY OF THE INVENTION

Provided herein are pluripotent adult stem cells (PASCs) that are c-kit positive and/or SSEA-4 positive. In some embodiments, the cells are also positive for one or more of the following: Oct-4, CD73, CD44, CD29 and CD105. In some embodiments, cells are CD133 negative. In some embodiments, cells are maternal cells isolated from placental tissue. In other embodiments, cells are isolated from endometrial tissue.

Methods of producing a population of cells enriched for pluripotent adult stem cells (PASCs) are also provided, including: selecting c-kit positive cells from an adult tissue (e.g., maternal placental tissue or endometrial tissue).

Additionally, methods of producing a population of cells enriched for pluripotent adult stem cells (PASCs) are provided, which include: selecting SSEA-4 positive cells from adult cells, e.g., maternal cells isolated from placental tissue, cells isolated from endometrial tissue, etc. In some embodiments, cells are selected for c-kit positive cells, either before or after said step of selecting SSEA-4 positive cells.

Further, methods of producing a population of cells enriched for pluripotent adult stem cells (PASCs) are provided, including: selecting SSEA-4 positive and c-kit positive cells from an adult tissue sample. In some embodiments, the sample is a placental tissue sample. In other embodiments, the sample is an endometrial tissue sample.

Methods of harvesting pluripotent adult stem cells (PASCs) are provided, including: providing full-term placenta tissue; collecting maternal cells from said full-term placenta tissue to provide a tissue sample; and selecting SSEA-4 positive cells from said tissue sample. In some embodiments, the methods also include the step of selecting c-kit positive cells, either before or after said selecting SSEA-4 positive cells. In some embodiments, collecting includes processing said placental tissue by proteolytic enzyme digestion. In some embodiments, the full-term placenta tissue includes decidua tissue.

Populations of cells consisting essentially of cells produced by the above methods are also provided. In some embodiments, the cells are positive for at least one of a marker selected from the group consisting of: Oct-4, CD73, CD44, CD29 and C105. In some embodiments, cells are negative for the marker CD133.

Methods of differentiating stem cells are provided, including: providing a population of cells described herein and inducing differentiation of said population of cells by exposing said cells to one or more differentiation-inducing agents, wherein said population are differentiated into cells selected from the group consisting of: osteogenic, hematopoietic, adipogenic, myogenic, hepatic, neurogenic and endothelial cells.

Methods of treating a subject in need thereof are provided, including: providing a population of cells described herein; inducing differentiation of said population of cells by exposing said cells to one or more differentiation-inducing agents, wherein said population are differentiated into cells selected from the group consisting of: osteogenic, hematopoietic, adipogenic, myogenic, hepatic, neurogenic and endothelial cells, to produce a population of differentiated cells; and administering the population of differentiated cells to the subject in need thereof.

Methods of treating a subject in need thereof are also provided, including: administering a population of cells described herein. In some embodiments, the subject is in need of cell replacement therapy. In some embodiments, the subject is in need of treatment for a spinal cord injury or neurodegenerative disease.

A further aspect of the present invention is the use of PASCs as described above for the preparation of a medicament for carrying out a method of treatment as described above, e.g., for cell replacement therapy, treatment of spinal cord injury, treatment of neurodegenerative disease, etc.

Methods for detecting the presence or absence of PASCs in a population of cells collected from an adult tissue or fluid are also provided. In some embodiments, the methods include the steps of: providing the population of cells; immunostaining the population of cells to detect one or more cells that are positive for one or more markers of interest; and optionally, karyotyping one or more cells that are positive for the markers of interest. In some embodiments, the markers of interest includes c-kit and/or SSEA-4. In some embodiments, the methods further include the step of determining the relative number of cells present in the population that are positive for the one or more makers of interest. In some embodiments, the adult tissue or fluid is a placental or endometrial tissue.

The foregoing and other objects and aspects of the present invention are explained in greater detail in the drawings herein and the specification set forth below.

### BRIEF DESCRIPTION OF THE DRAWINGS

FIG. 1. Cells isolated from placenta (Left) and c-kit selected cells from placenta (Right).

FIG. 2. The initial population of cells was characterized by immunophenotyping using a broad panel of antibodies including several hematopoietic, mesenchymal and progenitor markers. FIG. 3. The expression of Oct-4 and CD117 (c-kit) was demonstrated in all initial cell populations.

FIG. 4. Adipogenic differentiation. A: Control, B: Maternal placental cells. Osteogenic differentiation. C: Control; D: Maternal placental cells.

FIG. 5. Karyotype analysis shows that the cells are maternal in origin (the newborn was male).

### DETAILED DESCRIPTION OF THE PREFERRED EMBODIMENTS

Disclosed herein are pluripotent stem cells isolated from adult tissues and methods for isolation, propagation and differentiation of those cells. In some embodiments, pluripotent adult stem cells disclosed herein are phenotypically characterized by the detection of various markers, including, but not limited to, cell surface proteins and gene expression. In some embodiments, pluripotent adult stem cells are isolated by selecting for specific markers.

“Pluripotent adult stem cell” or “PASO” refers to a cell, or progeny of a cell, that (a) is found in, or is collected from, an adult tissue or fluid from a mammalian donor, (b) is pluripotent, (c) has substantial proliferative potential, (d) optionally, but preferably, does not require feeder cell layers to grow in vitro, (e) optionally, but preferably, specifically binds c-kit antibodies (particularly at the time of collection, as the ability of the cells to bind c-kit antibodies may be lost over time as the cells are grown in vitro), and (f) optionally, but preferably, specifically binds SSEA antibodies (e.g., SSEA-4 in human cells, particularly at the time of collection, as the ability of the cells to bind SSEA-4 antibodies may be lost over time as the cells are grown in vitro). “Pluripotent” refers to cells that can be differentiated into more than one of the following tissue types: adipogenic, osteogenic, myogenic, endothelial, neurogenic and hepatic tissues.

“Adult stem cells” are stem cells that are naturally found among differentiated cells in a tissue, organ, or bodily fluid. In preferred embodiments, adult stem cells are collected from a non-embryonic and non-fetal organism, i.e., post-birth, including organisms of any age, e.g., infant, juvenile, adolescent, adult and geriatric organisms.

“Isolated” as used herein signifies that the cells are placed into conditions other than their natural environment. The term “isolated” does not preclude the use of these cells thereafter in combinations or mixtures with other cells.

The disclosures of all cited United States Patent references are hereby incorporated by reference to the extent that they are consistent with the disclosures herein. As used herein in the description of the invention and the appended claims, the singular forms “a,” “an” and “the” are intended to include the plural forms as well, unless the context clearly indicates otherwise. Furthermore, the terms “about” and “approximately” as used herein when referring to a measurable value such as an amount of a compound, dose, time, temperature, and the like, is meant to encompass variations of 20%, 10%, 5%, 1%, 0.5%, or even 0.1% of the specified amount. Also, as used herein, “and/or” and “/” refer to and encompass any and all possible combinations of one or more of the associated listed items, as well as the lack of combinations when interpreted in the alternative (“or”).

### 1. Collection and Selection of Cells.

In general, pluripotent adult stem cells (PASCs) are cells, or progeny of cells, that are isolated from tissues or fluid, primarily mammalian tissues or fluid. The tissue or fluid may be obtained by, e.g., a tissue sampling technique, such as biopsy. Standard biopsy techniques known in the art may be employed.

In some embodiments, the cells are collected from placental tissues. In some embodiments, the placenta is hemochorial (i.e., maternal blood directly contacts fetal chorion, found in, e.g., human, monkey, mouse, rat, rabbit, etc.). In other embodiments the placenta is endothelialchorial (found in, e.g., dogs and cats), and in further embodiments the placenta is epitheliochorial (found in, e.g., horses, swine and ruminants). In some embodiments, placental tissues are full-term placental tissues, e.g., delivered during the third stage of childbirth or removed during a c-section delivery.

The placenta is made up of both fetal and maternal components. The fetal component is termed the chorion, and the maternal component is termed the decidua, or decidua basalis. The placenta, which is delivered at childbirth (e.g., during the third stage of labor during vaginal delivery or removed during caesarian section), typically contains both fetal and maternal tissues. For example, when the placenta separates from the endometrium during the third stage of labor it typically does not split between the maternal and fetal tissues. Rather, the placenta separates within the decidua, such that the delivered placental contains both fetal and maternal components. Therefore in some embodiments cells from the maternal component of a delivered placenta (i.e., the decidua) are harvested.

In some embodiments, a sample of placental tissue (e.g., from the decidua) may be obtained or harvested using, e.g., a punch-biopsy, a scalpel or homogenizing the placenta or a portion thereof using, for example, a blender. The homogenate may then be used as a source of cells. Other examples of methods of processing placental tissues are found in U.S. Application Publication No. 2008/0064098 to Allickson, which is incorporated by reference herein. In addition, kits for the collection of cord blood and placental tissues are available for the convenient collection of these tissues after the birth of a child (See, e.g., U.S. Pat. No. 7,147,626 to Goodman et al.). In some embodiments, collected and optionally processed placental tissues may be subsequently sent to stem cell banks or registries.

In further embodiments, cells are collected from endometrial tissues. The endometrium is the inner membrane, or lining, of the mammalian uterus. The endometrium grows to a thick, blood vessel-rich, glandular tissue layer during the menstrual cycle (in humans and the great apes) or estrous cycle (most other mammals) in order to become an optimal environment for the implantation of a blastocyst to initiate pregnancy. If no blastocyst is detected by the body, progesterone levels drop, and the endometrial lining is shed (in the menstrual cycle) or resorbed (in the estrous cycle). If a blastocyst is detected, the endometrial lining remains as the decidua, becoming the maternal portion of the placenta.

In some embodiments, a sample of endometrial tissue may be obtained or harvested using, e.g., a punch-biopsy of the tissue from the uterus, collection of tissue shedding during menstruation, etc. In other embodiments, hormones (such as progesterone or estrogen) may be administered to a subject or live tissue donor. in order to promote the growth of the endometrium prior to harvest. The tissue may, optionally, be homogenized using, for example, a blender. The homogenate may then be used as a source of cells. In some embodiments, collected and optionally processed endometrial tissues may be subsequently sent to stem cell banks or registries.

In some embodiments, tissues may be processed to facilitate selection for PASCs. For example, the collected or harvested tissue may be digested with proteolytic enzymes. Examples of proteolytic enzymes (also known as proteases) include, but are not limited to, calpain, carboxypeptidase, caspase, cathepsin, chymopapain, chymase, chymotrypsin, clostripain, collagenase, cucumisin, dispase, elastase, endoproteinase, enterokinase, proteasome, trypsin, etc. Cells may also be processed using a sequential digestion, e.g., dispase->trypsin or dispase->trypsin->collagenase I.

In further embodiments, cells collected may be characterized and/or sorted based upon whether they are maternal or fetal cells (see, e.g., U.S. Pat. No. 5,447,842 to Simons). Characterization to determine whether the cells are maternal or fetal may be performed by any conventional method in the art, e.g., karyotyping.

In some embodiments, markers are detected using a suitable immunological technique, c.g., flow cytomctry for membrane-bound markers, immunohistochemistry for intracellular markers, and enzyme-linked immunoassay for markers secreted into the medium. The expression of protein markers can also be detected at the mRNA level by, e.g., reverse transcriptase-PCR using marker-specific primers. See, e.g., U.S. Pat. No. 5,843,780.

From the tissue or fluid sample, PASCs may be isolated by selecting cells for a particular marker, in accordance with known techniques such as affinity binding and/or cell sorting. Markers may be surface markers, intracellular markers or secreted proteins. In some embodiments the cells are selected by cell sorting (e.g., FACS) with antibodies that specifically bind to desired markers.

Certain cell markers are thought to be particular to embryonic stem (ES) cells, as opposed to adult stem (AS) cells. For example, stage-specific embryonic antigens

(SSEA) are carbohydrate antigens that are characteristic of certain ES cell types. SSEA-1 appears during late cleavage stages of mouse embryos, and it is expressed by undifferentiated murine embryonic stem cells (Solter, 1978; Gooi, 1981). The expression of SSEA-1 decreases in murine embryonic stem cells upon differentiation, while SSEA-3 and SSEA-4 expression is typically increased (Solter, 1979). In contrast, undifferentiated human embryonic stem (hES) cells typically express SSEA-3 and SSEA-4 but not SSEA-1, and SSEA-3 and SSEA-4 expression decreases while SSEA-1 expression increases upon embryonic development (Andrews, 1984; Fenderson et al., 1987). Preimplantation human embryonic stem (hES) cells are typically SSEA-1 negative and SSEA-3/SSEA-4 positive. Human embryonic germ (hEG) cells are typically SSEA-1 positive. The differentiation of hES cells in vitro typically results in the loss of SSEA-3/SSEA-4 expression and increased expression of SSEA-1.

However, disclosed herein are adult stem cells, PASCs, that express SSEA antigens. Accordingly, in some embodiments, cells are selected for SSEA-4 expression (e.g., when pluripotent human stem cells are desired), and in other embodiments, cells are selected for SSEA-1 (e.g., when pluripotent murine stem cells are desired), based upon the species of origin of the cells. Antibodies useful in selecting for SSEA markers are available from, e.g., the Developmental Studies Hybridoma Bank (University of Iowa, Iowa City).

In some embodiments of the present invention, PASCs are also Oct-4 positive. Oct-4 is a POU transcription factor (Pesce et al., 1998) that is typically highly expressed in undifferentiated ES cells. The level of Oct-4 expression typically decreases upon stem cell differentiation.

Further embodiments of the present invention provide PASCs that are c-kit positive. The “c-kit” or “CD117” gene encodes a tyrosine kinase growth factor receptor for Stem Cell Factor (SCF) that is essential for hematopoiesis, melanogenesis and fertility. The c-kit receptor protein, also known as the “Steel factor receptor” or “stem cell factor receptor,” is constitutively expressed in hematopoietic stem cells, mast cells, germ cells, melanocytes, certain basal epithelial cells, luminal epithelium of breast, and the interstitial cells of Cajal of the gastrointestinal tract.

C-kit antibodies are known (see, e.g., U.S. Pat. Nos. 6,403,559, 6,001,803, and 5,545,533). Examples of c-kit antibodies include, but are not limited to, SCF (N-19), c-Kit (C-19), c-Kit (M-14), c-Kit (Ab 81), c-Kit (C-14), c-Kit (104D2), c-Kit (H-300), and c-Kit (E-1), which are all available from Santa Cruz Biotechnology, Inc. The c-Kit (E-1) antibody is a mouse monoclonal IgG recognizing an epitope corresponding to amino acids 23-322 mapping near the c-kit N-terminus and recognizes both c-Kit of human origin by both western blotting and immunohistochemistry. Other examples of antibodies that are commercially available and methods of production of c-kit antibodies are found in U.S. Patent Application Publication US 2005/0124003 to Atala et al., which is incorporated by reference herein. The use of cell surface antigens provides a means for the positive selection of certain stem cell populations, as well as for the phenotypic analysis of progenitor cell populations using, for example, immunoselection by flow cytometry. In some embodiments, cells selected for expression of c-kit and/or SSEA antigens may be further purified by selection for other stem cell and progenitor cell markers (e.g., Oct-4). The c-kit and/or SSEA positive cell selection can be accomplished by any suitable means known in the art, including flow cytometry, such as by fluorescence activated cell sorting (FACS) using a fluorochrome conjugated antibody. The selection may also be by high-gradient magnetic selection using antibody that is conjugated to magnetic particles, e.g., magnetic cell sorting (MACS). Any other suitable method, including attachment to and disattachment from a solid phase, is also contemplated.

In some embodiments PASCs may also be selected for expression of other markers, e.g., hematopoietic, mesenchymal and/or progenitor markers such as CD73, CD44, CD34, CD29, CD105, or combinations thereof, in a similar fashion as described above for c-kit and SSEA-4 positive cells. In further embodiments, cells may also be selected based upon being negative for a marker (e.g., CD133 negative) by conventional techniques (e.g., flow cytometry). These are known markers of embryonic and adult stem cells and indicate the pluripotential capacity of stem cells. These markers that can be used for immuno-isolation of the stem cells from a heterogeneous cell population.

Procedures for separation may include magnetic separation, using antibody-coated magnetic beads, affinity chromatography and “panning” with antibody attached to a solid matrix (e.g., a plate), etc. Fluorescence activated cell sorters can have varying degrees of sophistication, such as multiple color channels, low angle and obtuse light scattering detecting channels, impedance channels, etc. Dead cells may be eliminated by selection with dyes associated with dead cells (propidium iodide (PI), LDS). Any technique may be employed that is not unduly detrimental to the viability of the selected cells.

In some embodiments, the antibodies are conjugated with labels to allow for ease of separation of the particular cell type, e.g., magnetic beads; biotin, which binds with high affinity to avidin or streptavidin; fluorochromes, which can be used with a fluorescence activated cell sorter (FACS); haptens; etc. Multi-color analyses may be employed with the FACS or in a combination of immunomagnetic separation and flow cytometry. Multi-color analysis may be used for the separation of cells based on multiple surface antigens. Fluorochromes that find use in a multi-color analysis include phycobiliproteins, e.g. phycoeryirin and allophycocyanins; fluorescein and Texas red.

In some embodiments, the antibody (e.g., c-kit and/or SSEA antibody) is added to a cell sample. The amount of antibody necessary to bind a particular cell subset may be empirically determined by performing a test separation and analysis. The cells and antibodies are incubated for a period of time sufficient for complexes to form. The cells may additionally be incubated with antibodies or binding molecules specific for cell surface markers known to be present or absent on PASCs. For example, cells expressing certain markers can be negatively selected for.

The purified cell population may be collected in any appropriate medium. Various media are commercially available and may be used, including Dulbecco's Modified Eagle Medium (DMEM), Hank's Basic Salt Solution (HBSS), Dulbecco's phosphate buffered saline (dPBS), RPMI, Iscove's modified Dulbecco's medium (IMDM), phosphate buffered saline (PBS) with 5 mM EDTA, etc., frequently supplemented with fetal calf serum (FCS), bovine serum albumin (BSA), human serum albumin (HSA), etc.

Populations enriched for the PASCs described herein can be achieved in this manner. By “enriched” it is meant that the desired cells will be 30% or more of the cell composition, preferably 50% or more of the cell population, more preferably 90% or more of the cell population, and most preferably 95% or more of the cell population, as compared to the number of non-PASC cells present in the population. This does not include cells present solely for the support of the population, such as feeder cells. The relative number of cells present in a population may be determined by any conventional means, e.g., immunostaining for the presence or absence of PASC markers.

Alternatively, populations may consist essentially of PASCs described herein, such that the cell composition comprises at least 25%, 30%, 35%, 40%, 45% or 50% of PASCs. Further embodiments consist essentially of at least 60%, 70%, 75%, 80%, 85%, 90 or 95% of PASCs, as compared to the number of non-PASC cell types present in the population. Again, this does not include cells present solely for the support of the population, such as feeder cells, and the relative number of cells present in a population may be determined by any conventional means, e.g., immunostaining for the presence or absence of PASC markers.

### 2. Characterization of Cells.

The in vitro cell cultures described herein containing an enriched population of c-kit and/or SSEA positive PASCs are generally characterized in that the cultures stain positive for c-kit and/or SSEA (e.g., SSEA-4), produce progeny cells that can differentiate into at least two, preferably three, and most preferably more than three of the following cell lineages: osteogenic, adipogenic, neurogenic, myogenic, hematopoietic, hepatic and endothelial cell lineages in the presence of differentiation-inducing conditions. See, e.g., De Coppi et al., Nature Biotechnology (2007) 25(1): p. 100-6; PCT Application WO 03/042405 to Atala and DeCoppi.

In some embodiments, the population of cells contain at least 30% c-kit positive (c-kit+ or c-kit^{pos}) PASCs, preferably at least 50-70% c-kit+ PASCs, and more preferably greater than 80 or 90% c-kit+ cells. Most preferable would be a substantially pure population of c-kit+ cells, comprising at least 95% c-kit+ cells.

In some embodiments, the population of cells contain at least 30% SSEA positive (e.g., SSEA-4 positive) PASCs, preferably at least 50-70% SSEA positive cells, and more preferably greater thin 80 or 90% SSEA positive PASCs.

“Expression” of a gene encoding a specific marker means that the gene is transcribed, and preferably, translated. Typically, according to the present invention, expression of a gene encoding a specific marker will result in production of the encoded polypeptide.

The number of c-kit and/or SSEA positive cells in a cell population can be determined in any well known method known to one skilled in the art. For example, FACS analysis can be used. Alternatively, magnetic cell sorting technology (MACS) can be used to separate cells. In MACS, the c-kit and/or SSEA positive cells are specifically labeled with super-paramagnetic MACS MicroBeads, which can be designed to bind to either the c-kit antigen directly or indirectly. After magnetic labeling, the cells are passed through a separation column that is placed in a strong permanent magnet. The column matrix serves to create a high-gradient magnetic field. The magnetically labeled cells are retained in the column while non-labeled cells pass through. After removal of the column from the magnetic, field, the magnetically retained cells are eluted. Both labeled and non-labeled fractions can be recovered.

In some embodiments the PASCs express several markers characteristic of ES cells and/or various multipotent adult stem cells. In some embodiments PASCs are positive for at least one marker selected from the following: CD73, CD44, CD34, CD29 and CD105. In some embodiments the PASCs are negative for CD133. As those skilled in the art will appreciate, certain markers that were found in the initial populations of PASCs may be lost as the cells are passaged or differentiated, and certain markers may be gained.

Assays may be performed on cell lysates, intact cells, frozen sections, etc. In addition, selection of cells with preferred phenotypes as described herein may be selected for marker expression (e.g., CD73, CD44, CD34, CD29, CD105, or combinations thereof) in a similar fashion as described above for c-kit and SSEA positive cells. Cells may also be selected based upon being negative for a marker (e.g., CD133 negative) by conventional techniques (e.g., flow cytometry).

### 3. Propagation and Differentiation of Cells.

The “primary culture” is the first culture to become established after seeding disaggregated cells or primary explants into a culture vessel. “Propagating” or “expanding” as used herein refers to an increase in number of viable cells. Expanding may be accomplished by, e.g., “growing” the cells through one or more cell cycles, wherein at least a portion of the cells divide to produce additional cells.

“Passaged in vitro” or “passaged” refers to the transfer or subculture of a cell culture to a second culture vessel, usually implying mechanical or enzymatic disaggregation, reseeding, and often division into two or more daughter cultures, depending upon the rate of proliferation. If the population is selected for a particular genotype or phenotype, the culture becomes a “cell strain” upon subculture, i.e., the culture is homogeneous and possesses desirable characteristics.

Preferably, the PASCs are characterized by the ability to be grown in vitro without the need for feeder cells (Sce, e.g., WO 03/042405 to Atala and DeCoppi). In preferred embodiments undifferentiated PASCs stop proliferating when grown to confluence in vivo.

When desired, differentiation of cells to various cell types can be carried out in accordance with any of a variety of known techniques. Stem cells “differentiate” when they become more specialized cells. A variety of cell differentiation inducing agents can be used to differentiate the PASCs of the present invention into different phenotypes. To determine the differentiation status of the stem cells, the phenotypic characteristic of the cells can be observed using conventional methods such as light microscopy to detect cell morphology, RT-PCR to detect cell lineage specific transcription, and immunocytochemistry to detect markers specifically expressed in a particulate cell lineage.

In some embodiments the PASCs may be expanded in the presence of an agent that suppresses cellular differentiation, as known in the art (see Dushnik-Levinson et al., Biol. Neonate (1995) 67:77-83). Examples of agents that suppress cellular differentiation include leukemia inhibitory factor (LIF) and stem cell factor. Alternatively, agents may be used to induce diffenentiation, such as hydrocortisone, Ca²⁺, keratinocyte growth factor (KGF), TGF-β, retinoic acid, insulin, prolactin, sodium butyrate, TPA, DIVISO, NMF, DMF, collagen, laminin, heparan SO₄, androgen, estrogen, and combinations thereof (Culture of Epithelial Cells (1992) R. Ian Freshney ed., Wiley-Liss).

The cells may be assessed for viability, proliferation potential, and longevity using standard techniques in the art. For example, a trypan blue exclusion assay, a fluorescein diacetate uptake assay, a propidium iodide uptake assay, or other techniques known in the art may be used to assess viability. A thymidine uptake assay, an MTT cell proliferation assay, or other techniques known in the art may be used to assess proliferation. Longevity may be determined by, e.g., the maximum number of population doublings in extended cultures.

Additionally, cells of different lineages may be derived by inducing differentiation of PASCs and as evidenced by changes in cellular antigens. Various differentiation-inducing agents are used to accomplish such differentiation, such as growth factors (for example EGF, aFGF, bFGF, PIDGF, TGF-P), hormones (including but not limited to insulin, triiodothyronine, hydrocortisone, and dexamethasone), cytokines (for example IL-1α or P, IFN-γ, TFN), matrix elements (for example collagen, laminin, heparan sulfate, Matrigel), retinoic acid, transferrin, TPA, and DMSO. Such differentiation-inducing agents are known to those of ordinary skill in the art (Culture of Epithelial. Cells, (R. Ian Freshney ed., Wiley-Liss 1992)). Examples below describe differentiation of PASCs into osteogenic, adipogenic, myogenic and endothelial lineages. Identification of differentiated cells may be accomplished by staining the cells with tissue-specific antibodies according to techniques known in the art.

Upon appropriate stimulation, PASCs can be differentiated into various cell types. In some embodiments, PASCs are differentiated as follows: Osteogenic induction: culture in DMEN low glucose with 10% FBS supplementing with 100 nM dexamethasone (Sigma-Aldrich), 10 mM beta-glycerophosphate (Sigma-Aldrich) and 0.05 mM ascorbic acid-2-phosphate (Wako Chemicals, Irving, Tex.); Adipogenic induction: culture cells seeded at density of 3000 cells/cm² in DMEN low glucose medium with 10% FBS supplemented with 1 μM dexamethasone, 1 mM 3-isobutyl-1-methylxantine, 10 μg/ml insulin and 60 μM indomethacin (all from Sigma-Aldrich); Myogenic induction: plate cells into Matrigel-precoated dish (1 mg/ml, Collaborative Biomedical Products) and culture in myogenic medium (DMEN low glucose supplemented with 10% horse serum, and 0.5% chick embryo extract from Gibco), and follow by treatment of 5-azacytidine (10 μM, Sigma) added in myogenic medium for 24 h; Endothelial induction: plate cells into gelatin-precoated dish and cultured in endothelial basal medium-2 (EBM-2, Clonetics BioWittaker) supplemented with 10% FBS and 1% glutamine (Gibco). In some embodiments no feeder layer or leukaemia inhibitory factor (LIF) is required either for expansion or maintenance of PASCs in the culture process. Other examples of conditions that may be used to differentiate PASCs according to some embodiments can be found in U.S. Patent Application No. 2005/0124003 to Atala et al.

In some embodiment PASCs also show proliferative potential. For example, in some embodiments they proliferate through at least 60, 65, 70, 75 or 80 or more population doublings when grown in vitro. In some embodiments, PASCs proliferate through 100, 200 or 300 population doublings or more when grown in vitro.

In vitro growth conditions in some embodiments are the following: (a) placing of the tissue or other crude cell-containing fraction from a mammalian source onto a 24 well Petri dish a culture medium [α-MEM (Gibco) containing 15% ES-FBS, 1% glutamine and 1% Pen/Strept from Gibco supplemented with 18% Chang B and 2% Chang C from Irvine Scientific], upon which the cells are grown to the confluence, (b) dissociating the cells by 0.05% trypsin/EDTA (Gibco), (c) isolating an PASC subpopulation based on expression of a cell marker c-Kit using mini-MACS (Mitenyl Biotec Inc.), (d) plating of cells onto a Petri dish at a density of 3-8×10³/cm², and (e) maintaining the cells in culture medium until they reach 60% confluence in the dish.

In another embodiment the differentiating step is carried out by transfecting (also referred to as “engineering” or “transforming” or “transducing”) the cells with a vector that contains a nucleic acid encoding a differentiation factor (such as Pdx1, Ngn3, Nkx6.1, Nkx2.1, Pax6, or Pax4) and from which the differentiation factor can be expressed in the cells, or by activating the expression of an endogeneous nucleic acid encoding a differentiation factor in the cells (e.g., engineering the cells to activate transcription of an endogeneous differentiation factor such as Pdx1, Ngn3, Nkx6.1, Nkx2.1, Pax6, or Pax4, such as by inserting a heterologous promoter in operative association with an endogeneous differentiation factor, in accordance with known techniques. See, e.g., U.S. Pat. No. 5,618,698). Such exogeneous nucleic acids may be of any suitable source, typically mammalian, including but not limited to, rodent (mouse, hamster, rat), dog, cat, primate (human, monkey), etc. For recombinant techniques any suitable vector may be used, including plasmids, cosmids, bacteriophages, DNA viruses, RNA viruses and retroviruses, all of which are known for the expression of a heterologous nucleic acid in stem cells, progenitor cells, etc., in substantially the same manner as known. See, e.g., U.S. Pat. Nos. 6,392,118; 6,309,883; 6,258,354; and 4,959,313. Such adenovirus vectors are also known and can be utilized with PASCs as described herein in accordance with known techniques (See, e.g., U.S. Pat. Nos. 6,544,780; 6,503,498; 5,981,225; and 5,670,488). The vector should include a suitable promoter (such as an SV40 promoter, retrovirus LTR-promoter, or cytomegalovirus (CMV) promoter), operatively associated with the nucleic acid to constitutively express, or inducibly express, the differentiation factor in the cells. Expression may be stable expression or transient expression depending upon the specific system chosen.

If desired, the cells can be frozen or cryopreserved prior to use, and then thawed to a viable form. Methods of freezing or cryopreserving cells for subsequent return to viable form are well known in the art. For example, cryopreservation of cells can involve freezing the cells in a mixture of a growth medium and another liquid that prevents water from forming ice crystals, and then storing the cells at liquid nitrogen temperatures (e.g., from about −80 to about −196° C.). See, e.g., U.S. Pat. No. 6,783,964 to Opara.

### 4. Therapeutic Formulations and Methods of Treatment.

“Treat” as used herein refers to any type of treatment that imparts a benefit to a patient, e.g., a patient afflicted with or at risk for developing a disease. Treating includes actions taken and actions refrained from being taken for the purpose of improving the condition of the patient (e.g., the relief of one or more symptoms), delay in the onset or progression of the disease, etc.

Numerous diseases are characterized by the loss of function and/or loss of specific cell populations that are not regenerated by the body. Diseases such as neurodegenerative disorders and Type I diabetes, and ailments such as stroke, burns and other injuries may be treated with PASCs, differentiated or undifferentiated.

“Pharmaceutically acceptable” as used herein means that the compound or composition is suitable for administration to a subject to achieve the treatments described herein, without unduly deleterious side effects in light of the severity of the disease and necessity of the treatment.

“Subjects” as used herein are generally human subjects and include, but are not limited to, “patients.” The subjects may be male or female and may be of any race or ethnicity, including, but not limited to, Caucasian, African-American, African, Asian, Hispanic, Indian, etc. The subjects may be of any age, including fetal, newborn, neonate, infant, child, adolescent, adult, and geriatric.

Subjects may also include animal subjects, particularly mammalian subjects such as canines, felines, bovines, caprines, equines, ovines, porcines, rodents (e.g. rats and mice), lagomorphs, primates (including non-human primates), etc., for, e.g., veterinary medicine and/or pharmaceutical drug development purposes.

PASCs can be autogeneic (i.e., from the subject to be treated), isogeneic (i.e., a genetically identical but different subject, such as from an identical twin), allogeneic (i.e., from a non-genetically identical member of the same species) or xenogeneic (i.e., from a member of a different species).

Diseases related to the lack of a particular secreted product such as hormone, enzyme, growth factor, or the like may be treated with PASCs according to some embodiments. For example, CNS disorders encompass numerous afflictions such as neurodegenerative diseases (e.g. Alzheimer's and Parkinson's), acute brain injury (e.g. stroke, head injury, cerebral palsy) and a large number of CNS dysfunctions (e.g. depression, epilepsy, and schizophrenia). In recent years neurodegenerative disease has become an important concern due to the expanding elderly population, which is at greatest risk for these disorders. These diseases, which include Alzheimer's Disease, Multiple Sclerosis (MS), Huntington's Disease, Amyotrophic Lateral Sclerosis, and Parkinson's Disease, have been linked to the degeneration of neural cells in particular locations of the CNS, leading to the inability of these cells or the brain region to carry out their intended function. By providing for maturation, proliferation and differentiation into one or more selected lineages through specific different growth factors, PASCs may be used as a source of committed or differentiated cells for therapeutic administration.

PASCs may also be used in enzyme replacement therapy in specific conditions including, but not limited to, lysosomal storage diseases, such as Tay-Sachs, Niemann-Pick, Fabry's, Gaucher's, Hunter's, Hurler's syndrome, as well as other gangliosidoses, mucopolysaccharidoses, and glycogenoses.

Additionally, PASCs of the present invention may be used as transgene carriers in gene therapy to correct inborn errors of metabolism affecting the cardiovascular, respiratory, gastrointestinal, reproductive, and nervous systems, or to treat cancer and other pathological conditions.

In some embodiment PASCs can be used in tissue regeneration/replacement therapy. PASCs of the present invention may also be used in reconstructive treatment of damaged tissue by surgical implantation of cell sheets, disaggregated cells, and cells embedded in carriers for regeneration of tissues for which differentiated cells have been produced. In some embodiments cells are used in tissue engineered constructs. Such constructs may include a biocompatible polymer formed into a scaffold suitable for cell growth. The scaffold can be shaped into, e.g., a heart valve, vessel (tubular), planar construct or any other suitable shape. Such constructs are known in the art (see, e.g., WO02/035992, U.S. Pat. Nos. 6,479,064, 6,461,628). Cells may be differentiated before or after seeding.

Formulations including PASCs, differentiated or undifferentiated, include those for parenteral administration (e.g., subcutaneous, intramuscular, intradermal, intravenous, intraartcrial, intraperitoneal injection) or implantation. In one embodiment, administration is carried out intravascularly, either by simple injection, or by injection through a catheter positioned in a suitable blood vessel, such as a renal artery. In some embodiments, administration is carried out by “infusion,” whereby compositions are introduced into the body through a vein (e.g., the portal vein). In another embodiment, administration is carried out as a graft to an organ or tissue to be augmented as discussed above, e.g., kidney and/or liver.

A “biodegradable scaffold or matrix” is any substance not having toxic or injurious effects on biological function and is capable of being broken down into is elemental components by a host. Preferably, the scaffold or matrix is porous to allow for cell deposition both on and in the pores of the matrix. Such formulations can be prepared by supplying at least one cell population to a biodegradable scaffold to seed the cell population on and/or into the scaffold. The seeded scaffold may then implanted in the body of a recipient subject.

The cells may be formulated for administration in a pharmaceutical carrier in accordance with known techniques. See, e.g., Hariri, U.S. Patent Application 2003/0180289; Remington, The Science And Practice of Pharmacy (9^{th} Ed. 1995). In the manufacture of a pharmaceutical formulation according to the invention, the cells are typically admixed with, inter alia, an acceptable carrier. The carrier must, of course, be acceptable in the sense of being compatible with any other ingredients in the formulation and must not be deleterious to the patient. The carrier may be a solid or a liquid, or both (e.g., hydrogels), and may be formulated with the cells as a unit-dose formulation. In one embodiment the cells are provided as a suspension in the carrier to reduce clumping of the cells.

In further embodiments, if desired or necessary, the subject may be administered an agent for inhibiting transplant rejection of the administered cells, such as rapamycin, azathioprine, corticosteroids, cyclosporin and/or FK506, in accordance with known techniques. See, e.g., R. Caine, U.S. Pat. Nos. 5,461,058, 5,403,833 and 5,100,899; see also U.S. Pat. Nos. 6,455,518, 6,346,243 and 5,321,043. Some embodiments use a combination of implantation and immunosuppression, which minimizes graft rejection. The implantation may be repeated as needed to create an adequate mass of transplanted tissue.

The present invention is explained in greater detail in the following non-limiting Examples.

### Examples

It is herein demonstrated that pluripotent adult stem cells can be obtained with CD117 isolation, and these cells are able to differentiate into various cell types.

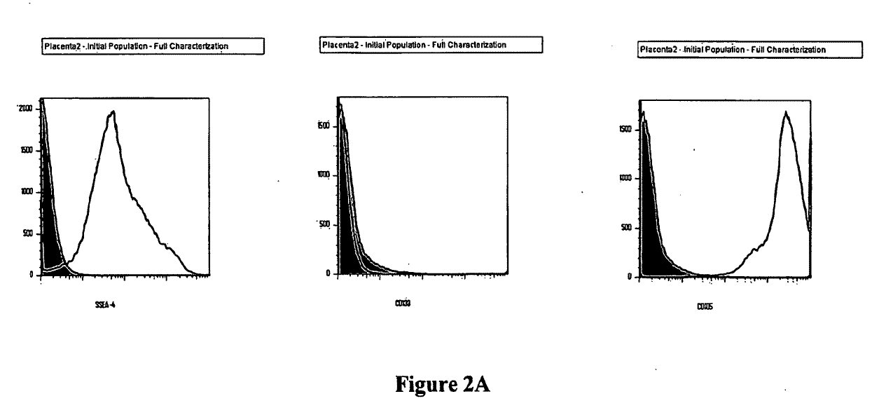

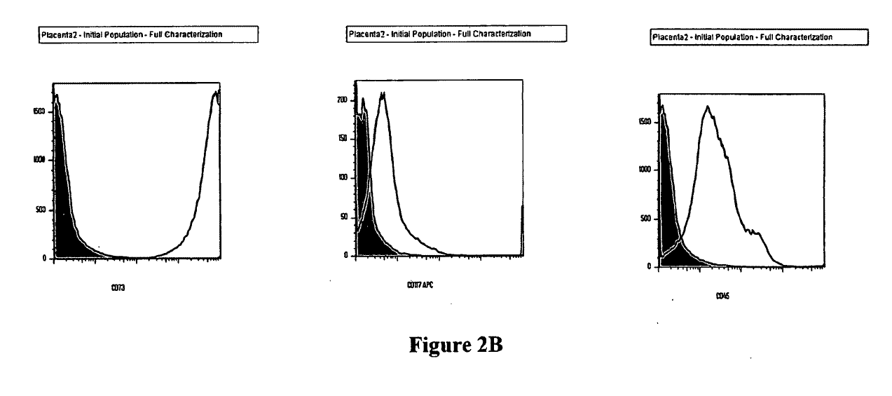

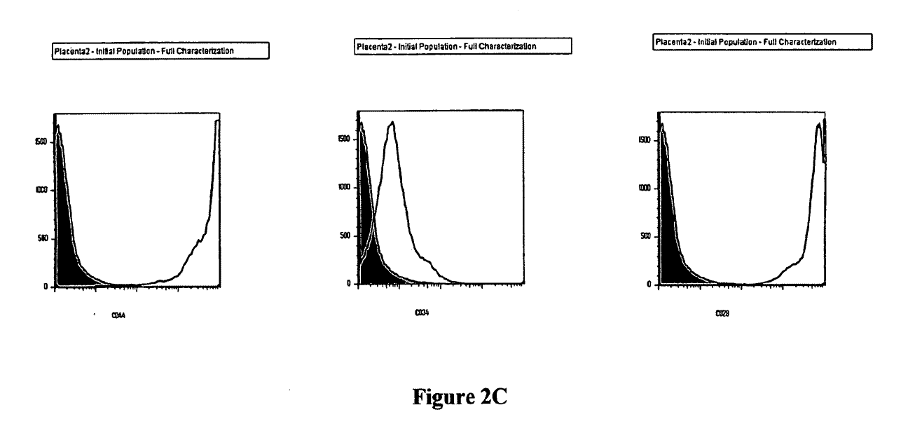

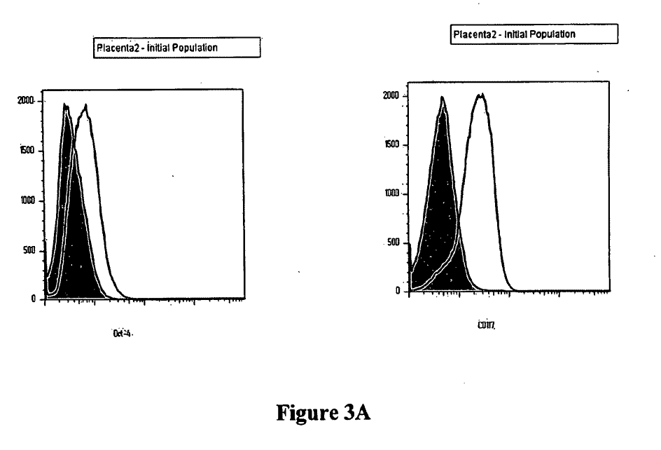

Cells were successfully isolated from human full-term placentas and processed using a sequential digestion with different proteolytic enzymes: dispase->trypsin->collagenase I. The initial population of cells were expanded and characterized by immunophenotyping using a broad panel of antibodies that included several hematopoietic, mesenchymal and progenitor markers (FIG. 2). The initial population contained a population of cells expressing nearly 80% of SSEA-4 positive cells. These cells expressed mesenchymal, hematopoietic and progenitor cell marker CD117. The cells also expressed CD73, CD44, CD29, and CD105, and were negative for CD133. All of the initial populations of cells were also analyzed for two particular stem cell markers, CD117 and Oct-4, and the expression of low levels of Oct-4 and CD117 was demonstrated in all initial cell populations that were tested (FIG. 3).

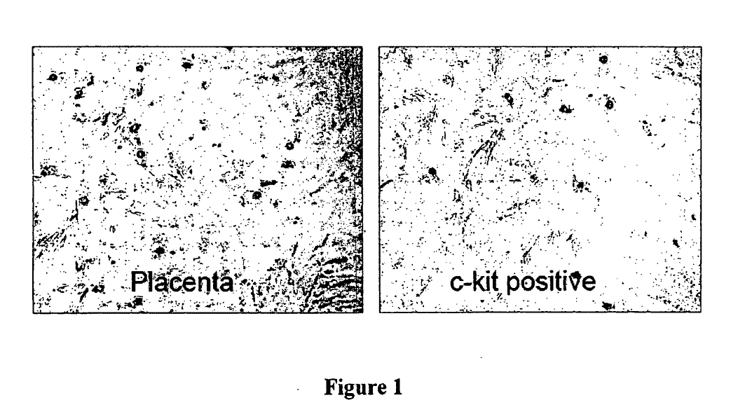

Initial isolation was followed by magnetic cell sorting using CD117 microbeads (Miltenyi Biotec) on both populations put together (trypsin and collagenase populations). An SSEA-4 magnetic cell sorting isolation was also accomplished on unselected placenta cells (i.e., no c-kit isolation). The c-kit sorted cells were similar to the cells in the initial populations with an increased homogeneity in the phenotype (FIG. 1).

Cloning by dilution was achieved using the CD117 sorted cells. After initial isolation and expansion, the clones were analyzed by flow cytometry for the multipotent marker Oct-4, to show low levels of Oct-4 expression. An established amniotic fluid stem cell line (A1) was used as the control for the flow cytometry analysis.

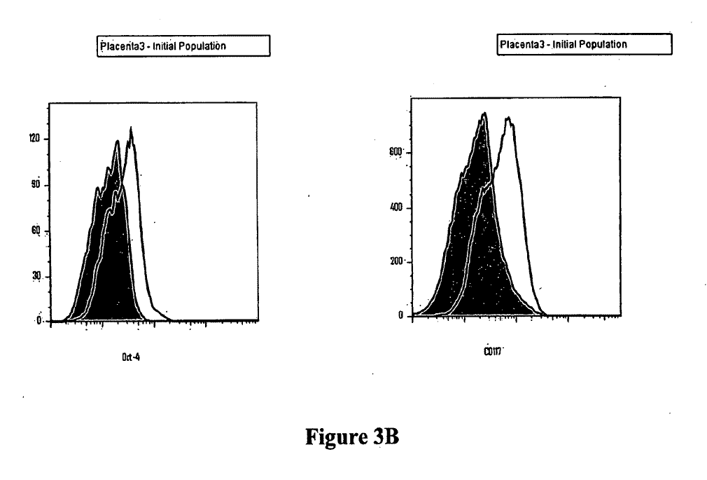

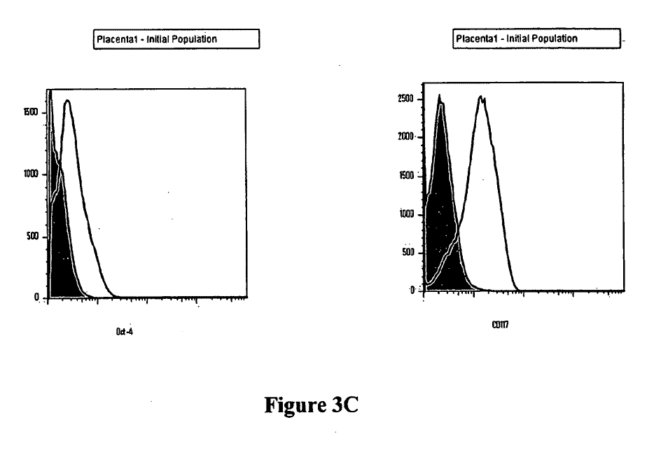

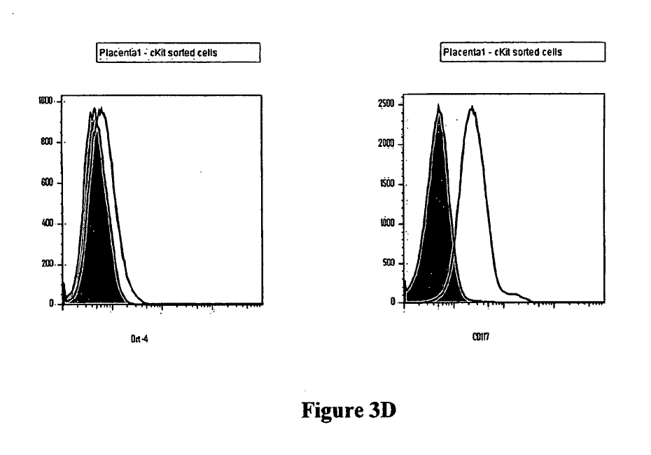

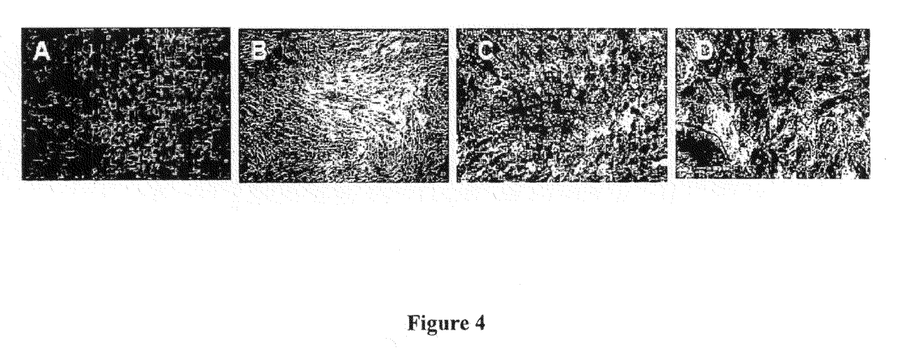

To demonstrate whether these cells have the capability to differentiate into various cell types, experiments were performed on the CD117 and SSEA-4 sorted cells. The initial populations were used as a control. Adipogenic and osteogenic differentiation was performed according to previously reported techniques (See U.S. Patent Application Publication No. 2005/0124003 to Atala et al.; De Coppi et al., Nature Biotechnology 25(1):100-106). Human bone marrow mesenchymal stem cells (hBM-MSC) were used as a positive control and PASC culture medium (α-MEM (Gibco) containing 15% ES-FBS, 1% glutamine and 1% Pen/Strept from Gibco supplemented with 18% Chang B Medium® and 2% Chang C Medium® (Irvine Scientific, Santa Ana, Calif.)) as negative experimental control in the hBM-MSC and placental clones. Complete differentiation was observed with hBM-MSC in both adipogenic and osteogenic lineages (FIG. 4).

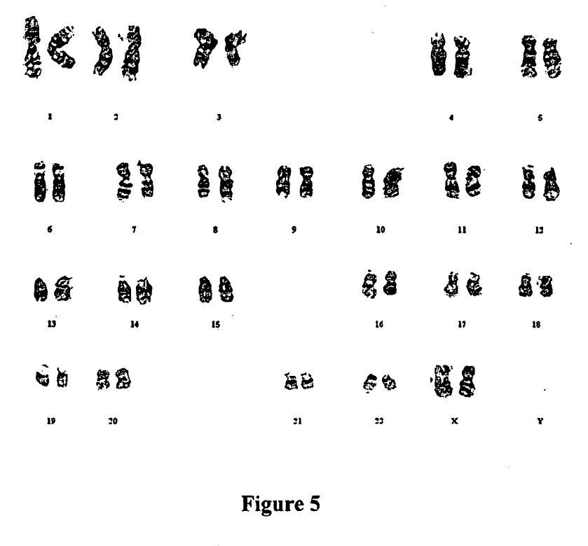

Karyotype analysis was performed to determine the source of the cells (FIG. 5). It was determined that these cells were female (the newborn was male), indicating the mother was the origin of the cells.

These data demonstrate that pluripotent adult stem cells (PASCs) can be isolated and that these cells have the capability of differentiating into other cell lineages when guided appropriately.

The foregoing is illustrative of the present invention, and is not to be construed as limiting thereof. The invention is defined by the following claims, with equivalents of the claims to be included therein.

1. An isolated pluripotent adult stem cell (PASC) that is c-kit positive and SSEA-4 positive.

2. The cell of claim 1, wherein said cell is Oct-4 positive.

3. The cell of claim 1, wherein said cell is CD73 positive.

4. The cell of any claim 1, wherein said cell is CD44 positive.

5. The cell of any of claim 1, wherein said cell is CD29 positive.

6. The cell of claim 1, wherein said cell is CD105 positive.

7. The cell of claim 1, wherein said cell is CD133 negative.

8. The cell of claim 1, wherein said cell is a maternal cell isolated from placental tissue.

9. The cell of claim 1, wherein said cell is isolated from endometrial tissue.

10. A population of cells consisting essentially of the cell of claim 1.

11. A method of producing a population of cells enriched for pluripotent adult stem cells (PASCs), comprising:
selecting c-kit positive cells from a sample, wherein said selected c-kit positive cells are maternal cells isolated from placental tissue;
to thereby produce a population of cells enriched for PASCs.

12. A method of producing a population of cells enriched for pluripotent adult stem cells (PASCs), comprising:
selecting c-kit positive cells from a sample, wherein said selected c-kit positive cells are isolated from endometrial tissue;
to thereby produce a population of cells enriched for PASCs.

13. A method of producing a population of cells enriched for pluripotent adult stem cells (PASCs), comprising:
selecting SSEA-4 positive cells from a sample, wherein said selected SSEA-4 positive cells are maternal cells isolated from placental tissue;
to thereby produce a population of cells enriched for PASCs.

14. A method of producing a population of cells enriched for pluripotent adult stem cells (PASCs), comprising:
selecting SSEA-4 positive cells from a sample, wherein said selected SSEA-4 positive cells are isolated from endometrial tissue;
to thereby produce a population of cells enriched for PASCs.

15. The method of claim 14, further comprising the step of selecting c-kit positive cells, either before or after said step of selecting SSEA-4 positive cells.

16. A population of cells consisting essentially of cells produced by the method of claim 11.

17. The population of claim 16, wherein said cells are positive for a marker selected from the group consisting of: Oct-4, CD73, CD44, CD29, C105, and combinations thereof.

18. The population of claim 17, wherein said cells are negative for the marker CD133.

19. A method of producing a population of cells enriched for pluripotent adult stem cells (PASCs), comprising:
selecting SSEA-4 positive and c-kit positive cells from an adult tissue sample; to
thereby produce a population of cells enriched for PASCs.

20. The method of claim 16, wherein said sample is a placental tissue sample.

21. The method of claim 16, wherein said sample is an endometrial tissue sample.

22. A population of cells consisting essentially of cells produced by the method of claim 19.

23. The population of claim 22, wherein said cells are positive for at least one of a marker selected from the group consisting of: Oct-4, CD73, CD44, CD29 and C105.

24. The population of claim 19, wherein said cells are negative for the marker CD133.

25. A method of harvesting pluripotent adult stem cells (PASCs) comprising:
providing full-term placenta tissue;
collecting maternal cells from said full-term placenta tissue to provide a tissue sample; and
selecting SSEA-4 positive cells from said tissue sample, to thereby harvest PASCs.

26. The method of claim 25, further comprising the step of selecting c-kit positive cells, either before or after said selecting SSEA-4 positive cells.

27. The method of claim 25, wherein said collecting comprises processing said placental tissue by proteolytic enzyme digestion.

28. The method of claim 25, wherein said full-term placenta tissue comprises decidua tissue.

29. A method of differentiating stem cells, comprising:
providing a population of cells according to claim 10; and
inducing differentiation of said population of cells by exposing said cells to one or more differentiation-inducing agents, wherein said population are differentiated into cells selected from the group consisting of: osteogenic, hematopoietic, adipogenic, myogenic, hepatic, neurogenic and endothelial cells;
to thereby differentiate said stem cells.

30. A method of treating a subject in need thereof, comprising:
providing a population of cells according to claim 10;
inducing differentiation of said population of cells by exposing said cells to one or more differentiation-inducing agents, wherein said population are differentiated into cells selected from the group consisting of: osteogenic, hematopoietic, adipogenic, myogenic, hepatic, neurogenic and endothelial cells, to produce a population of differentiated cells; and
administering the population of differentiated cells to said subject in need thereof.

31. The method of claim 30, wherein said subject is in need of cell replacement therapy.

32. The method of claim 30, wherein said subject is in need of treatment for a spinal cord injury or neurodegenerative disease.

33. A method of treating a subject in need thereof, comprising:
administering a population of cells according to claim 10.

34. The method of claim 33, wherein said subject is in need of cell replacement therapy.

35. The method of claim 33, wherein said subject is in need of treatment for a spinal cord injury or neurodegenerative disease.

36-38. (canceled)

39. A method for detecting the presence or absence of a pluripotent adult stem cell (PASC) in a population of cells collected from an adult tissue or fluid, said method comprising the steps of:
providing said population of cells;
immunostaining said population of cells to detect one or more cells that are positive for one or more markers of interest; and
optionally, karyotyping one or more cells that are positive for said markers of interest.

40. The method of claim 39, wherein said one or more markers of interest includes c-kit.

41. The method of claim 39, wherein said one or more markers of interest includes SSEA-4.

42. The method of claim 39, further comprising the step of determining the relative number of cells present in said population that are positive for said one or more makers of interest.

43. The method of claim 39, wherein said adult tissue or fluid is a placental or endometrial tissue.
